(19) United States
(12) Patent Application Publication
(10) Pub. No.: US 2011/0172404 A1
(43) Pub. Date: Jul. 14, 2011

(54) Self-Assembly of Nanoparticles Through Nuclei Acid Engineering

(75) Inventors: Dan Luo, Ithaca, NY (US); Wenlong Cheng, Victoria (AU)

(73) Assignee: Cornell University, Ithaca, NY (US)

(21) Appl. No.: 12/993,231

(22) PCT Filed: May 19, 2009
(86) PCT No.: PCT/US2009/044526

§ 371 Date: Apr. 4, 2011

Related U.S. Application Data
(60) Provisional application No. 61/054,334, filed on May 19, 2008.

(51) Int. Cl. C07H 21/00; C07H 21/02; C07H 21/04; C07H 1/00; B82Y 5/00; B82Y 40/00

(52) U.S. Cl. 536/23.1; 977/773; 977/810; 977/774; 977/795; 977/840

(57) ABSTRACT

A self-assembly nanodevice formed through nucleic acid engineering is disclosed. The nanodevice may include an array of nanoparticles. The nanodevice may further include a substrate that supports the array of nanoparticles. Each of the nanoparticles may be coordinated with a plurality of nucleic acids that are substantially free of Watson-Crick base-paring with nucleic acids coordinated with other nanoparticles. Methods of forming the nanodevice, as well as the microscopic organization of the nanoparticles are also disclosed. By manipulating the nucleic acids as capping ligands, the inter-particle distance may be extended to a greater range than nanotechnology based on alkyl ligands or nucleic acids base-pairing.

### CROSS-REFERENCE TO RELATED APPLICATION

This application is based on and claims priority from U.S. provisional Application Ser. No. 61/054,334, filed on May 19, 2008.

### BACKGROUND OF THE DISCLOSURE

1. Technical Field

A self-assembly nanodevice formed through nucleic acid engineering is disclosed. The nanodevice includes an array of nanoparticles each coordinated with a plurality of nucleic acids that are substantially free of Watson-Crick base-paring with nucleic acids coordinated with other nanoparticles. By changing the length and/or coordination number of the nucleic acids, the inter-particle distances may be manipulated within a significantly wider range than that achieve by using alkylthiol as the ligands to cap the nanoparticles.

2. Description of the Related Art

Development of nanotechnology focusing on the control of matter on an atomic and molecular scale has gained significant interest in recent decades. In general, nanotechnology deals with structures having sizes of 100 nanometers or smaller, and involves developing materials or devices within that size. Nanotechnology is very diverse, ranging from novel extensions of conventional device physics, to completely new approaches based upon molecular self-assembly, to developing new materials with dimensions on a nanoscale.

Molecular self-assembly is an important aspect of bottom-up approaches to nanotechnology. Using molecular self-assembly the final (desired) structure is programmed in the shape and functional groups of the molecules. Self-assembly is referred to as a ‘bottom-up’ manufacturing technique in contrast to a ‘top-down’ technique such as lithography where the desired final structure is carved from a larger block of matter. In the speculative vision of molecular nanotechnology, microchips of the future might be made by molecular self-assembly. An advantage to constructing nanostructure using molecular self-assembly for biological materials is that they will degrade back into individual molecules that can be broken down by the body.

DNA nanotechnology is an area of current research that uses the bottom-up, self-assembly approach for molecular self-assembly. DNA nanotechnology uses the unique molecular recognition properties of DNA and other nucleic acids, e.g. Watson-Crick base-pairing, to create self-assembling branched DNA complexes with useful properties. DNA is thus used as a structural material rather than as a carrier of biological information, to make structures such as two-dimensional and three-dimensional lattice structures.

Transition metal nanoparticles, such as gold nanoparticles, have been the focus of intense interest recently due to their potential use in the fields of optics, immunodiagnostics, and electronics. The transition metal nanoparticles may exist in a variety of shapes including spheres, rods, cubes, and caps. In application, the transition metal nanoparticles are generally coordinated to, and stabilized by, a ligand.

In order to tailor properties of nanoparticles, three parameters should be considered: (1) particle morphology (usually refers to the particle size and shape) due to the so-called quantum size effects; (2) surface ligands that protect particles from agglomeration into bulk materials; and (3) 2D or 3D microscopic organization of the particles. In the past few years, extensive research efforts have been directed to the manipulation of these parameters in order to make self-assembled bottom-up structures or devices from nanoscale building blocks, instead of the current top-down lithographic techniques.

However, up to now a practical self-assembled device has not been realized yet. For example, there is not a generally applicable approach that addresses self-assembly of gold nanoparticles with full control over the above-mentioned three parameters. Specifically, it has been documented that nanoparticle supracrystals or metamaterials can be fabricated from gold nanoparticles surrounded by alkylthiol ligands. See, e.g. Courty, A., Mermet, A., Albouy, P. A., Duval, E., Pileni, M. P. Nat. Mater. 4, 395-398 (2005); Kiely, C. J., Fink, J., Brust, M., Bethel, D., Schiffrin, D. J., “Spontaneous ordering of bimodal ensembles of nanoscopic gold clusters,” Nature 396, 444-446 (1998); Boal, A. K., Ilhan, F., DeRouchey, J. E., Thum-Albrecht, T., Russell, T. P., Rotello, V. M., “Self-assembly of nanoparticles into structured spherical and network aggregates,” Nature 404, 746-748 (2000); Korgel, B. A. and Fitzmaurice, D., “Condensation of ordered nanocrystal thin films,” Phys. Rev. Lett. 80, 3531-3534 (1998); Bigioni, T. P., Lin, X,-M., Nguyen, T. T., Corwin, E., Witten, T. A., Jaeger, H. M., “Kinetically driven self assembly of highly ordered nanoparticle monolayers,” Nature Materials 5, 265-270 (2006); and Shevchenko, E. V., Talapin, D. V., Kotov, N. A., O'Brien, S., Murray, C, B., “Structural diversity of binary nanoparticle superlattices,” Nature 495, 55-59 (2006). Although alkylthiols appear to be good candidates to cap and organize gold nanoparticles, the use of alkylthiol as ligand appears to enable manipulation of interparticle distances within a relatively narrow range and the alkylthiol-capped nanoparticles are not water-soluble, which limits its application to self-assembled devices with biological systems.

Self-assembly of gold nanoparticles based on DNA nanotechnology has also been developed in recent years. See, e.g. Mirkin, C. A., Letsinger, R. L., Mucic, R. C., Storhoff, J. J., Nature 382, 607-609 (1996); Zheng, J. W., Constantinou, P. E., Micheel, C., Alivisatos, A. P., Kiehl, R. A., Seeman, N. C., “Two-Dimensional Nanoparticle Arrays Show the Organizational Power of Robust DNA Motifs,” Nano Lett., 6 (7), 1502-1504 (2006). In particular, a layer of “anchor” nucleic acid is deposited onto a target surface and contacted by gold nanoparticles conjugated with a layer of “probe” nucleic acid in a wetted process environment that favors Watson-Crick base-pairing between the “anchor” and “probe” nucleic acids to attach the gold nanoparticles to the target surface, forming an organized 2D or 3D lattice structure. To facilitate the attachment, each nanoparticle is coordinated with a relatively small number, e.g. about 60, of nucleic acids to prevent steric hindrance that disfavors Watson-Crick base-pairing. Under dewetted conditions, however, the organized lattice structure formed by Watson-Crick base-pairing collapse and as result, the desirable surface properties conferred by the nanoparticles are affected.

Hence, there is a need for forming an array of transition metal nanoparticles on a nanodevice with greater manipulability of interparticle distance. Moreover, there is a need for forming an array of transition metal nanoparticles without collapsing under dewetted conditions. Finally, there is a need for economical, convenient, and robust formation of well-defined arrays of nanoparticles in a nanodevice.

### SUMMARY OF THE DISCLOSURE

In satisfaction of the aforementioned needs, A self-assembly nanodevice formed through nucleic acid engineering is disclosed. The nanodevice includes an array of nanoparticles. The array of nanoparticles may be supported by a substrate. Each of the nanoparticles may be coordinated with a plurality of nucleic acids that are substantially free of Watson-Crick base-paring with nucleic acids coordinated with other nanoparticles. Methods of forming the nanodevice, as well as the microscopic organization of the nanoparticles on the substrate are also disclosed. By using and manipulating the nucleic acids as capping ligands, the interparticle distance (edge-to-edge) may be extend to a greater range than that achieved by using alkylthiol as the ligands to coordinate the nanoparticles.

The substrate that supports the nanoparticles may be an apparatus, device, material, or composite generally used in nanotechnology. In one embodiment, the substrate is a holey carbon film or silicon nitride film. In another embodiment, the substrate is a copper grid. The substrate may include holes, pores, webs, dents, recesses, grooves, or other regular or irregular surface features that provide support for the array of nanoparticles. Alternatively, the disclosed nanodevice may be substrate-free, in which the array of nanoparticles form a self-supported superlattice structure.

The nanoparticles suitable for use in this disclosure may comprise one or more transition metals. In particular, transition metals used in nanotechnology may include gold, silver, platinum, cadmium, etc. In one embodiment, the nanoparticles are gold nanoparticles. In addition to transition metals, the nanoparticles used in this disclosure may also include a quantum dot.

In order to form an organized array of nanoparticles, each of the nanoparticles may be coordinated with a plurality of nucleic acids, such as those in the form of DNAs, RNAs, PNAs, LNAs, GNAs, TNAs, and mixtures thereof. In one embodiment, the nucleic acids are DNAs selected from a group consisting of single-stranded DNAs, double-stranded DNAs, hairpin DNAs, dendrimer DNAs, quadruplex DNAs, and mixtures thereof. In another embodiment, the nucleic acids are single- or double-stranded oligonucleotides.

The molar ratio of the nucleic acids and nanoparticle may be at least 100:1. In one embodiment, the molar ratio of the nucleic acids and nanoparticle may be from about 200:1 to about 300:1. One feature of this disclosure is that the nucleic acids coordinated with each nanoparticle may be substantially free of Watson-Crick base-pairing with nucleic acids coordinated with other nanoparticles.

Unlike the use of alkylthiols as the capping ligand, the interparticle-distance of the nanoparticles according to this disclosure may be tuned from about 0.8 nm to about 50 nm by varying the length of the nucleic acids. In one embodiment, the interparticle-distance is from about 2 nm to about 27 nm. In yet another embodiment, the interparticle-distance is from about 2 to about 25 nm or even from about 3 nm to about 25 nm. The length of the nucleic acids, e.g. the average number of nucleotides in each single-stranded DNA, may be from about 5 to about 90 in some embodiments. In other embodiments, the length of the nucleic acid used in this disclosure may be from 5 to 160 or even from 5 to 200. For double-stranded DNAs, two Watson-Crick base-paired nucleotides only count as one towards the length of the nucleic acids.

To form the organized array of nanoparticles on the substrate, a dispersion or colloid of the nanoparticles coordinated with the nucleic acids in a liquid carrier is prepared. Because of the enhanced stability of the nanoparticles according to this disclosure, the dispersion may have a concentration up to about 82 mg/ml. The dispersion is dropped onto the substrate and dried under a dewetted condition. The liquid carrier may be an aqueous medium with properties that disfavors Watson-Crick base-pairing. In one embodiment, the liquid carrier comprises a low-salt buffer (<1 mM NaCl).

The array of nanoparticles on the substrate thus formed may be a 2D superlattice or a 3D crystal with an anisotropic optical response. In one embodiment, the array of nanoparticles has a well-organized, defect-free structure extending to a dimension of at least 10 μm.

The disclosed nucleic acids-coordinated nanoparticles may be processed into micro- and nano-scale patterns by PDMS microcontact printing. As a result, nanoscale features can be obtained through micrometer-sized molds. In one embodiment, PDMS surface pattern edges are nucleation sites of the nanoparticles to achieve line resolution with single particle size width. In another embodiment, the nanoparticles according to this disclosure are printable by a nanopen. In a refinement, micro-scale letters from gold nanoparticles can be obtained with a density of 9×10⁴/cm².

Other advantages and features of the disclosed methods and device will be described in greater detail below. It will also be noted here and elsewhere that the device or method disclosed herein may be suitably modified to be used in a wide variety of application by one of ordinary skill in the art without undue experimentation.

### BRIEF DESCRIPTION OF THE DRAWINGS

For a more complete understanding of the disclosed device and methods, reference should be made to the embodiments illustrated in greater detail in the accompanying drawings, wherein:

FIG. 1 is a schematic illustration of forming the array of nanoparticles in one embodiment of this disclosure;

FIG. 2A is a TEM micrograph of an array of gold nanoparticles (5′-thiol-ATGGCAACTATTTACGCGCTAGAGTCGT-3′ as ligands) supported by a porous lacey carbon film;

FIG. 2B is an expanded TEM micrograph of the array of gold nanoparticles illustrated in FIG. 2A;

FIG. 2C is an expanded TEM micrograph of the array of gold nanoparticles illustrated in FIG. 2B;

FIG. 3A is a TEM micrograph of an array of gold nanoparticles (5′-thiol-ATGGCAACTATTTACGCGCTAGAGTCGT-3′ as ligands) supported by a quantifoil holey carbon film with square holes (7×7 μm);

FIG. 3B is an expanded TEM micrograph of the array of gold nanoparticles illustrated in FIG. 3A;

FIG. 3C is an expanded TEM micrograph of the array of gold nanoparticles illustrated in FIG. 3B;

FIG. 4A is a TEM micrograph of an array of gold nanoparticles (5′-thiol-ATGGCAACTATTTACGCGCTAGAGTCGT-3′ as ligands) supported by a holey silicon nitride film (thickness of 50 nm);

FIG. 4B is an expanded TEM micrograph of the array of gold nanoparticles illustrated in FIG. 4A;

FIG. 5A illustrates a comparison between TEM micrographs of an array of gold nanoparticles (5′-SH-poly(dT)₅ as ligands) according to this disclosure and an array of gold nanoparticles organized by Watson-Crick base-pairing of nucleic acids with 5 nucleotides, both of which are supported by a 2000-mesh copper grid (7×7 μm holes);

FIG. 5B illustrates a comparison between TEM micrographs of an array of gold nanoparticles (5′-SH-poly(dT)₁₅ as ligands) according to this disclosure and an array of gold nanoparticles organized by Watson-Crick base-pairing of nucleic acids with 15 nucleotides, both of which are supported by a 2000-mesh copper grid (7×7 μm holes);

FIG. 5C illustrates a comparison between TEM micrographs of an array of gold nanoparticles (5′-SH-poly(dT)₃₀ as ligands) according to this disclosure and an array of gold nanoparticles organized by Watson-Crick base-pairing of nucleic acids with 30 nucleotides, both of which are supported by a 2000-mesh copper grid (7×7 μm holes);

FIG. 5D illustrates a comparison between TEM micrographs of an array of gold nanoparticles (5′-SH-poly(dT)₅₀ as ligands) according to this disclosure and an array of gold nanoparticles organized by Watson-Crick base-pairing of nucleic acids with 50 nucleotides, both of which are supported by a 2000-mesh copper grid (7×7 μm holes);

FIG. 5E illustrates a comparison between TEM micrographs of an array of gold nanoparticles (5′-SH-poly(dT)₇₀ as ligands) according to this disclosure and an array of gold nanoparticles organized by Watson-Crick base-pairing of nucleic acids with 70 nucleotides, both of which are supported by a 2000-mesh copper grid (7×7 μm holes);

FIG. 5F illustrates a comparison between TEM micrographs of an array of gold nanoparticles (5′-SH-poly(dT)₉₀ as ligands) according to this disclosure and an array of gold nanoparticles organized by Watson-Crick base-pairing of nucleic acids with 90 nucleotides, both of which are supported by a 2000-mesh copper grid (7×7 μm holes);

FIG. 6A illustrates a TEM micrograph and microabsorption spectrum of an array of gold nanoparticles (5′-SH-poly(dT)₅ as ligands) supported by a 2000-mesh copper grid (7×7 μm holes);

FIG. 6B illustrates a TEM micrograph and microabsorption spectrum of an array of gold nanoparticles (5′-SH-poly(dT)₃₀ as ligands) supported by a 2000-mesh copper grid (7×7 μm holes);

FIG. 6C illustrates a TEM micrograph and microabsorption spectrum of an array of gold nanoparticles (5′-SH-poly(dT)₉₀ as ligands) supported by a 2000-mesh copper grid (7×7 μm holes);

FIG. 7A is a TEM micrograph of an array of gold nanoparticles (5′-thiol-ATGGCAACTATTTACGCGCTAGAGTCGT-3′ as ligands) supported by a 2000-mesh copper grid (7×7 μm holes), wherein the molar ratio between the ligands and nanoparticle is 1000:1;

FIG. 7B is a TEM micrograph of an array of gold nanoparticles (5′-thiol-ATGGCAACTATTTACGCGCTAGAGTCGT-3′ as ligands) supported by a 2000-mesh copper grid (7×7 μm holes), wherein the molar ratio between the ligands and nanoparticle is 500:1;

FIG. 7C is a TEM micrograph of an array of gold nanoparticles (5′-thiol-ATGGCAACTATTTACGCGCTAGAGTCGT-3′ as ligands) supported by a 2000-mesh copper grid (7×7 μm holes), wherein the molar ratio between the ligands and nanoparticle is 100:1;

FIG. 7D is a TEM micrograph of an array of gold nanoparticles (5′-thiol-ATGGCAACTATTTACGCGCTAGAGTCGT-3′ as ligands) supported by a 2000-mesh copper grid (7×7 μm holes), wherein the molar ratio between the ligands and nanoparticle is 50:1;

FIG. 8 is a TEM micrograph of an array of gold nanoparticles (5′-SH-poly(dT)₅ as ligands) that forms 3D crystals on a silicon substrate; and

FIG. 9 is a TEM micrograph of a micro-disc of 2D gold nanoparticel superlattices (5′-thiol-ATGGCAACTATTTACGCGCTAGAGTCGT-3′ as ligands) formed by PDSM microcontact printing.

It should be understood that the drawings are not necessarily to scale and that the disclosed embodiments are sometimes illustrated diagrammatically and in partial views. In certain instances, details which are not necessary for an understanding of the disclosed device or method which render other details difficult to perceive may have been omitted. It should be understood, of course, that this disclosure is not limited to the particular embodiments illustrated herein.

### DETAILED DESCRIPTION OF THE DISCLOSURE

This disclosure is generally related to the use of nucleic acids as capping ligands to organize nanoparticles on a substrate surface in a dewetted self-assembly process without depending on Watson-Crick base-pairing. The edge-to-edge interparticle distances in this disclosure can be tuned from about 0.8 nm to about 50 nm, which is a significantly wider range than that achieved by the use of alkylthiols as capping ligands.

Material

Gold nanoparticles used herein are prepared following the process disclosed in Frens, G, “Controlled nucleation for the regulation of particle size in monodispersed gold suspension,” Nat. Phy. Sci. 241, 20-22 (1973). The gold nanoparticles have a diameter of 12.8±1.2 nm. However, it is to be understood that this disclosure is not limited to gold nanoparticles. Nor are the gold nanoparticles used in this disclosure limited to the aforementioned size and preparation process.

The nucleic acids used in this disclosure may be in the form of DNAs, RNAs, PNAs, LNAs, GNAs, TNAs, and mixtures thereof. In one embodiment, the nucleic acids are DNAs selected from a group consisting of single stranded DNAs, double stranded DNAs, hairpin DNAs, dendrimer DNAs, quadruplex DNAs, and mixtures thereof. In one embodiment, the nucleic acid ligands are thiolated oligonucleotides such as 5′-SH-poly(dT)ₓ, wherein x is an integer of 5-90. In another embodiment, the thiolated oligonucleotide is 5′-thiol-ATGGCAACTATTTACGCGCTAGAGTCGT-3′. The thiolated oligonucleotides may be purchased from Integrated DNA Technologies, 1710 Commercial Park, Coralville, Iowa 52241. It should be noted that this disclosure is not limited to the nucleic acids enumerated herein, other nucleic acids of different type, chemical composition, spatial configuration, etc. can also be used in accordance with this disclosure without undue experimentation.

In the disclosed non-limiting examples, the substrates can be obtained from commercial vendors. Specifically, holey Lancey/Formvar carbon films having irregular pores with sizes ranging from less than 0.25 micron to more than 10 micron can be obtained from Ted Pella, Inc, P.O. Box 492477, Redding, Calif. 96049-2477 (www.tedpella.com). Copper grids (2000 mesh) having a regular array of square holes (7×7 μm) can also be obtained from Ted Pella. In addition, C-flat holey carbon (circular holes of 1 μm diameter and 1 μm space), quantifoil holey carbon films (circular holes of 2 μm diameter and 1 μm space), and quantifoil holey carbon films (square holes of 7×7 μm) can also be obtained from Electron Microscopy Sciences, P.O. Box 550, 1560 Industry Road, Hatfield, Pa. 19440 (http://www.cmsdiasum.com/). Finally, holey silicon nitride films (circular holes of 2 μm diameter and 2 μm space) can be obtained from SPI Supplies, 569 East Gay Street, West Chester, Pa. 19381-0656 (http://www.2spi.com).

Synthesis of Gold Nanoparticles Capped with Nucleic Acids

Preparation of the Gold Nanoparticle-Nucleic Acids Complex is Based on a modification of the process disclosed in Thaxton, C. S., Hill, S. D., Geoganopoulou, D. G., Stoeva, S. I., Mirkin, C. A., “A bio-bar-code assay based upon dithiothreitol-induced oligonucleotide release,” Anal. Chem. 77, 8174-78 (2005).

In particular, thiolated oligonucleotides is reduced by DTT or TCEP and mixed with a solution of gold nanoparticles (molar ratio of oligonucleotide to nanoparticle molar is about 1000:1). The mixture is allowed to stand for 12 hours at room temperature, after which sodium chloride is added up to a concentration of about 1M. Then, the solution is aged for another 10-12 hours and centrifuged at 14500 g for 30 min to obtain a red precipitate, which is then redispersed in Mili-Q water to form a dispersion or colloid for subsequent self-assembly processes.

Self-Assembly of Gold Nanoparticles Capped with Nucleic Acids

The dispersion of gold nanoparticle-nucleic acids complex in Mili-Q water is dropped onto one or more holes of the substrates discussed above. The dispersion is subsequently dried to allow the nanoparticles to self-assemble into an organized 2D superlattice or 3D crystal. It is to be understood that the self-assembly of the nanoparticles coordinated with nucleic acids is not limited to the non-limiting exemplary process disclosed herein. Other processes or methods used in nanotechnology may also be used in view of this disclosure.

Microscopic Analysis of the Nanoparticles on the Substrate Surface

High-resolution structural characterization of the nanoparticle superlattices is carried out using a Tecnai T12 TEM (Transmission Electron Microscopy). Stepwise beam focusing with low beam currents is used to minimize distortion of the lattice structure by electronic beams. Microspectra of the nanoparticle arrays are obtained through a Renishaw Raman spectrometer that records local transmission spectra (˜1 μm area) after illuminating a sample with a Halogen white light source. The data recorded is then normalized to obtain microabsorption spectra of the nanoparticle arrays.

Characteristics of the Nanoparticle Self-Assembly

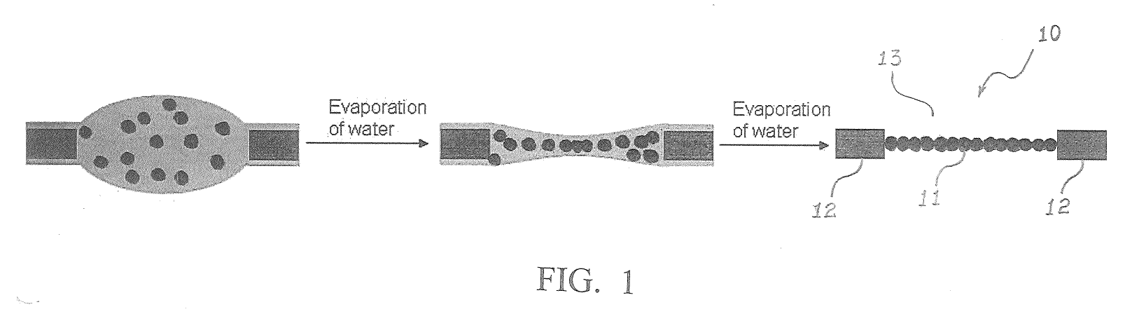

As schematically illustrated in FIG. 1, the disclosed nanodevice 10 may include an array of nanoparticles 11. Each nanoparticle 11 may be coordinated with a plurality of nucleic acids to regulate the interparticle distances within the nanodevice 10. The array of nanoparticles may be supported by a substrate 12. In the embodiment illustrated in FIG. 1, the array of nanoparticles is formed within a hole 13 of the substrate. However, the nanoparticles may also be coated on or otherwise supported by the substrate.

Without wishing to be bound by any particular theory, it is contemplated that the self-assembly of nanoparticles may occur during the drying of the dispersion containing nanoparticles coordinated with nucleic acids. The ability of the array of nanoparticles disclosed herein to withstand a dewetted condition allow for a more practical approach to form self-assembly nanoparticles through a convenient, economical, and robust process that is yet to be realized by existing methods and devices.

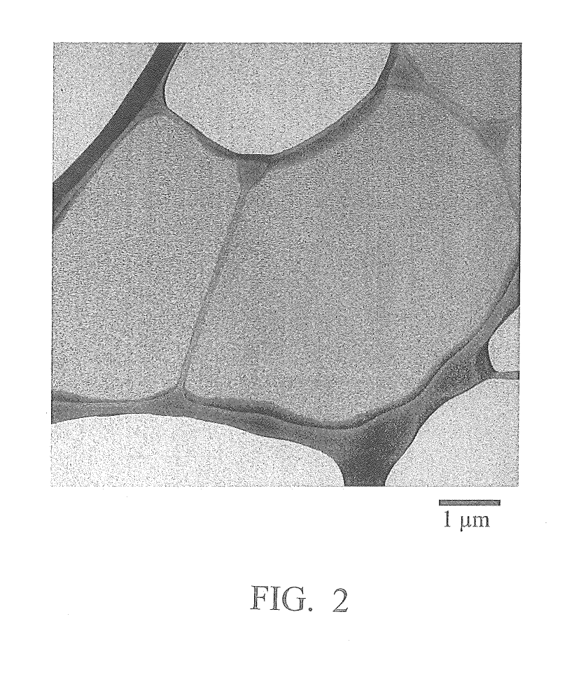

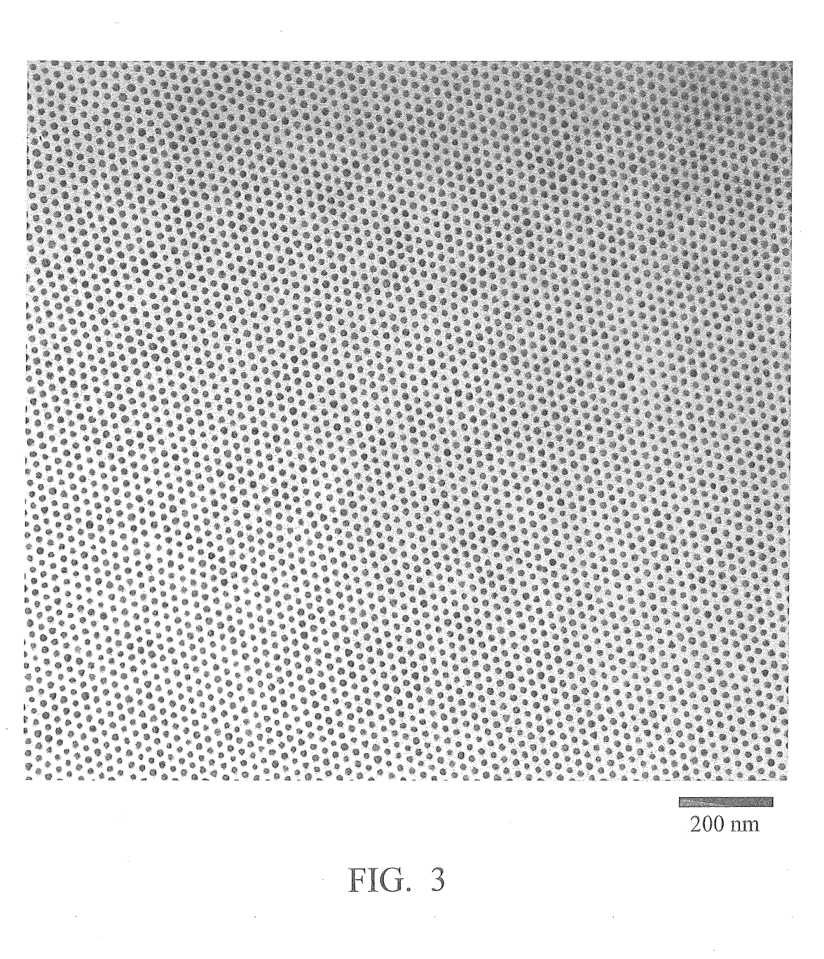

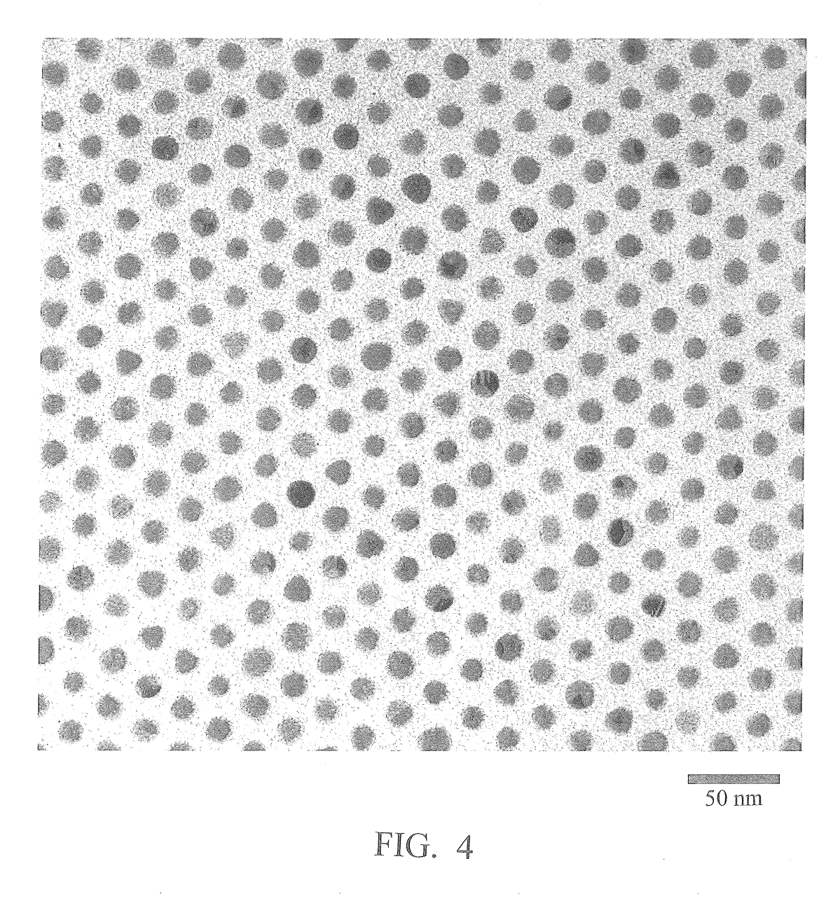

Turning to FIGS. 2A-2C, TEM micrographs of an array of gold nanoparticles (5′-thiol-ATGGCAACTATTTACGCGCTAGAGTCGT-3′ as ligands) according to one embodiment of this disclosure are illustrated. A well-organized 2D superlattice structure supported by a porous lacey carbon film is formed by the method disclosed herein.

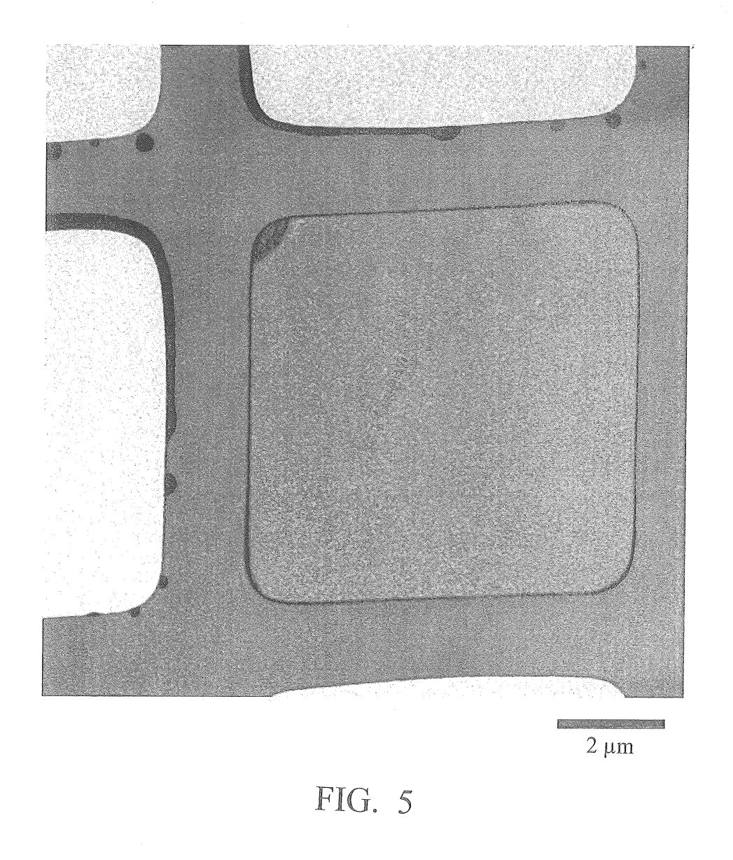

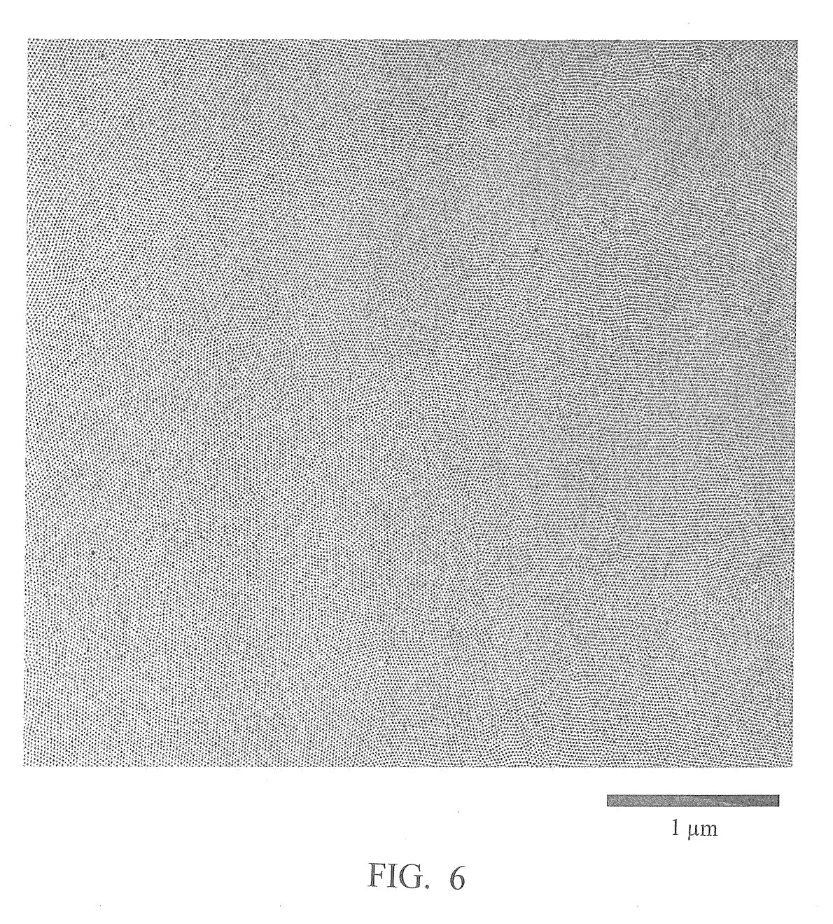

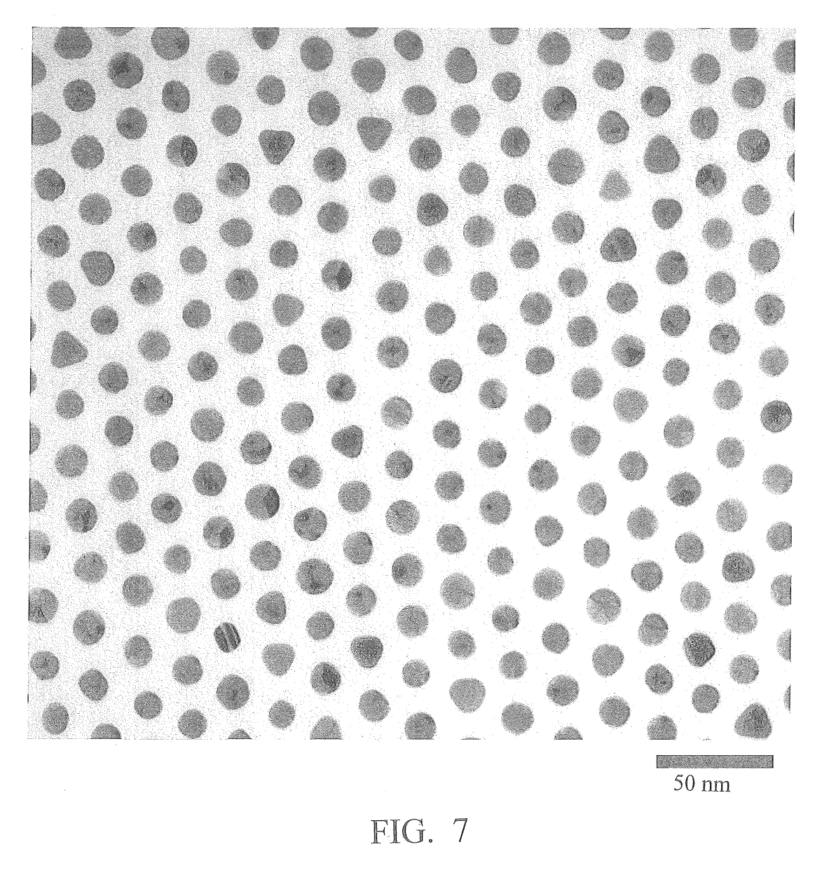

Similarly, FIGS. 3A-3C illustrate TEM micrographs of an array of gold nanoparticles (5′-thiol-ATGGCAACTATTTACGCGCTAGAGTCGT-3′ as ligands). Under this condition, a well-organized 2D superlattice structure supported by a quantifoil holey carbon film with square holes (7×7 μm) is also obtained.

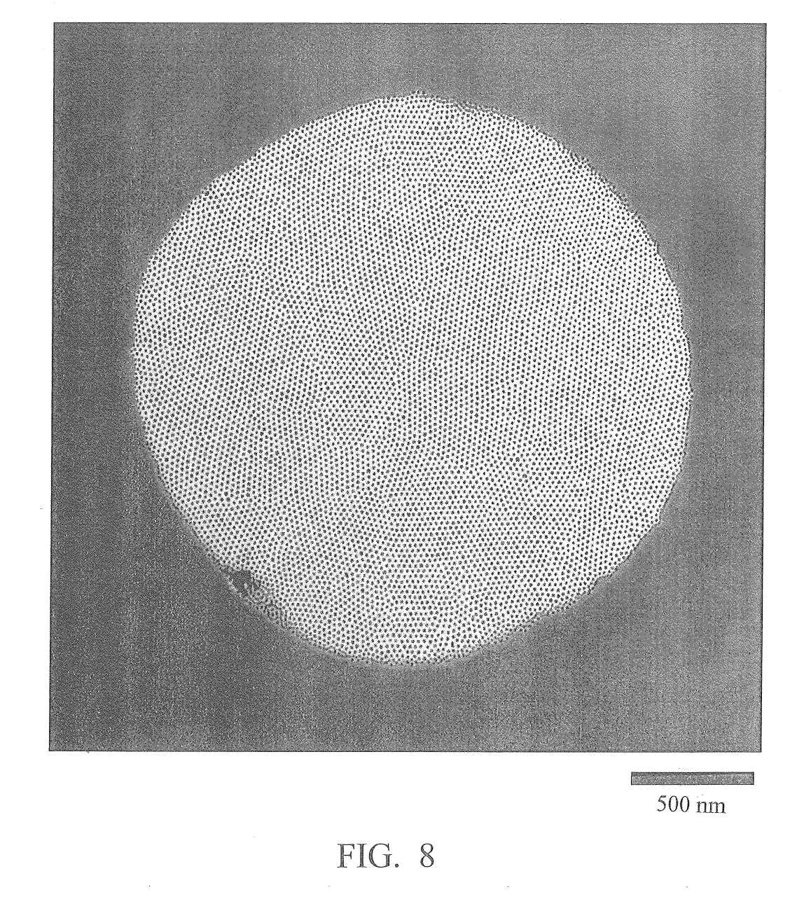

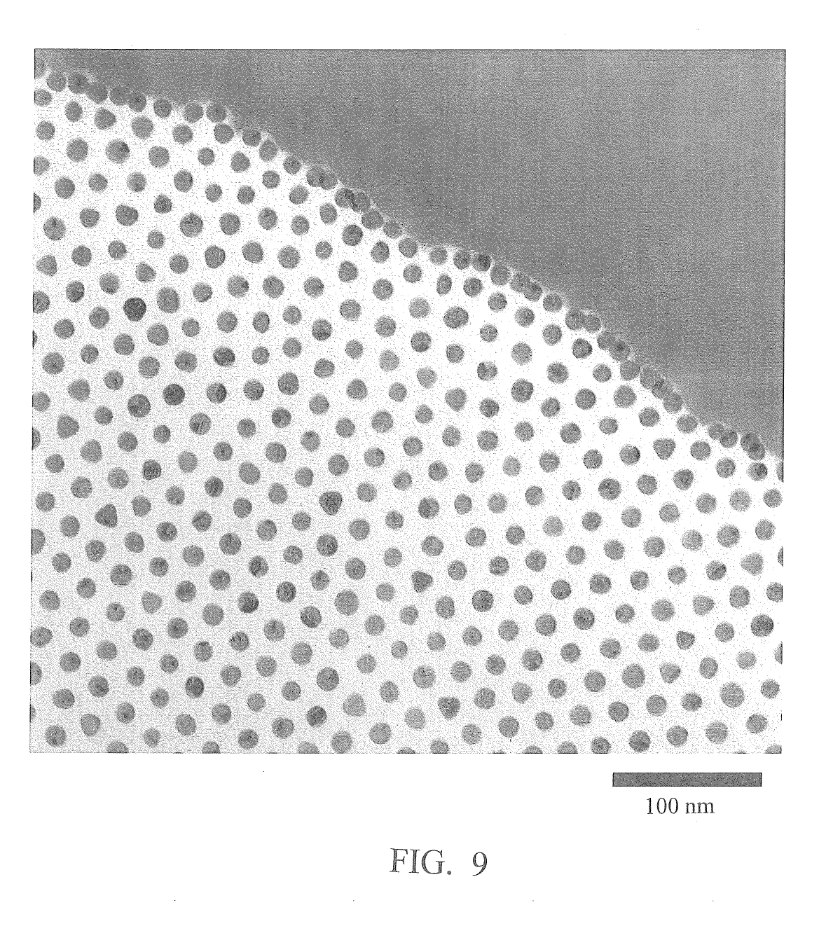

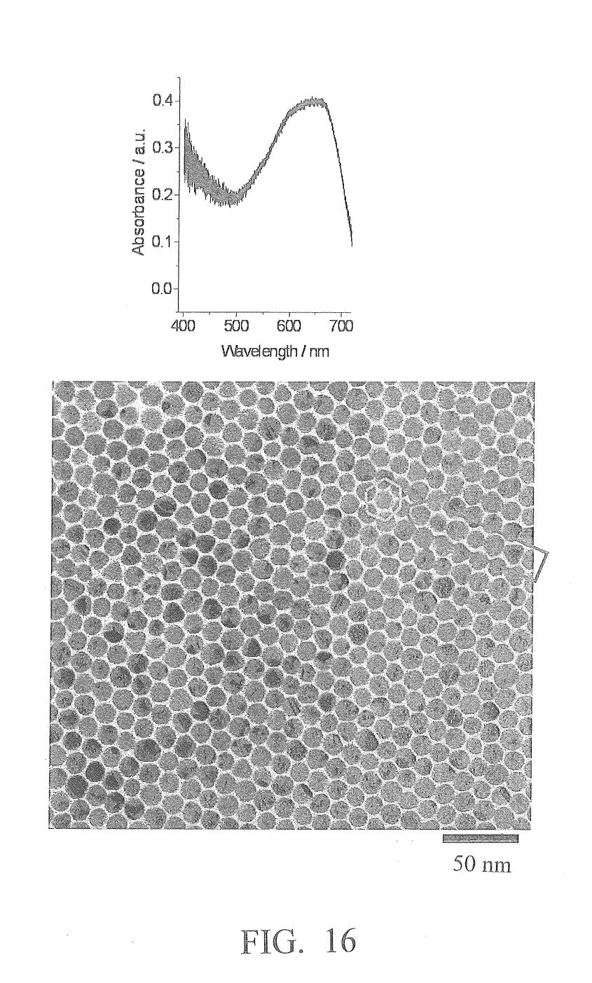

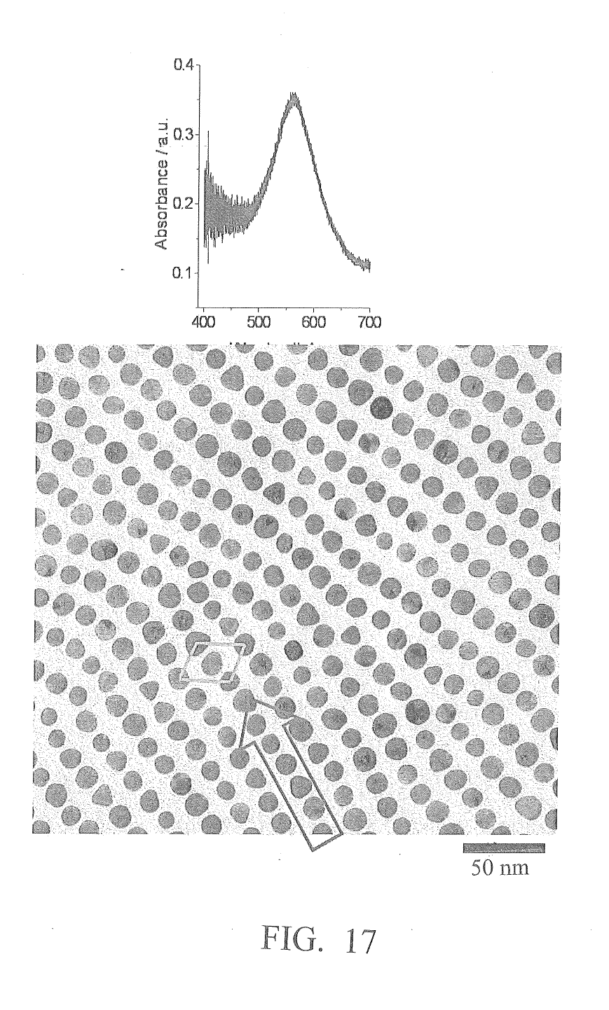

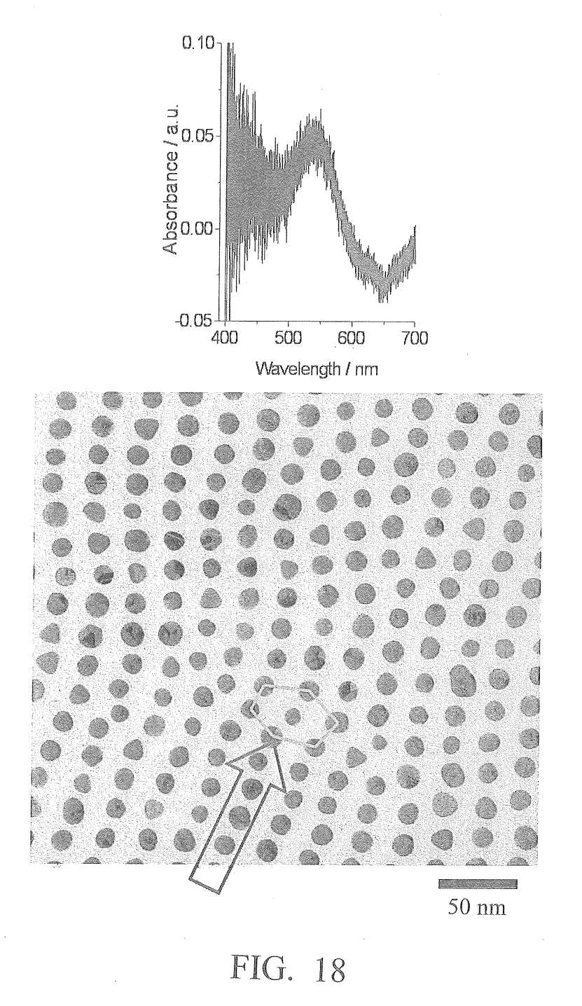

The substrate that supports the nanoparticle superlattice structure is not limited to carbon films. For example, as shown in FIG. 4A-4B, an array of gold nanoparticles (5′-thiol-ATGGCAACTATTTACGCGCTAGAGTCGT-3′ as ligands) supported by a holey silicon nitride film (thickness of 50 nm) may also be produced with a well-organized 2D superlattice structure. Copper grid may also be used to support the array of nanoparticles, as illustrated in FIGS. 6A-6C.

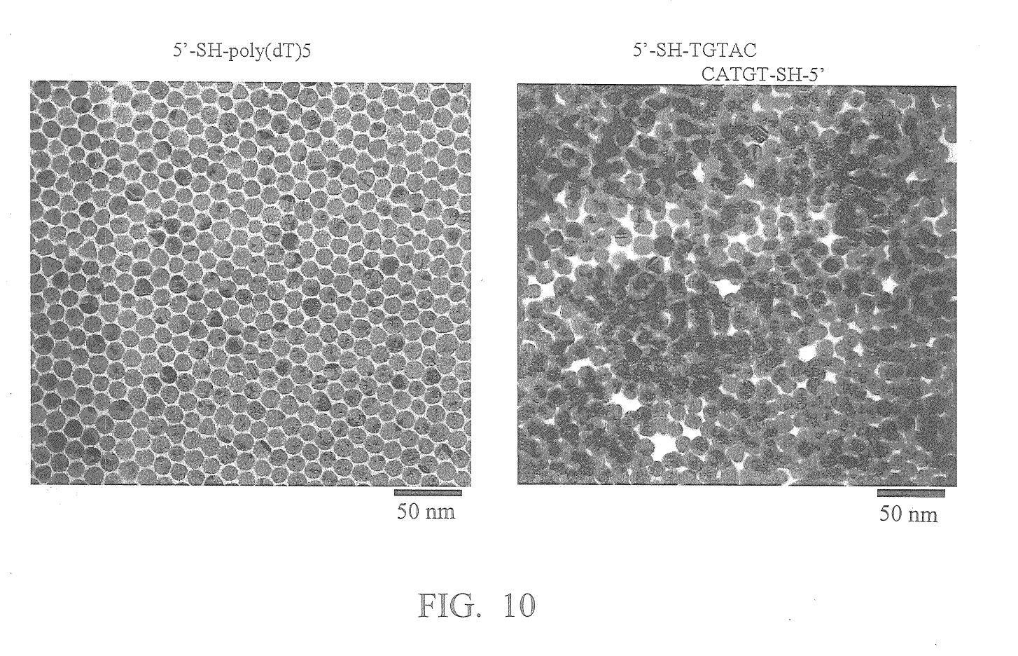

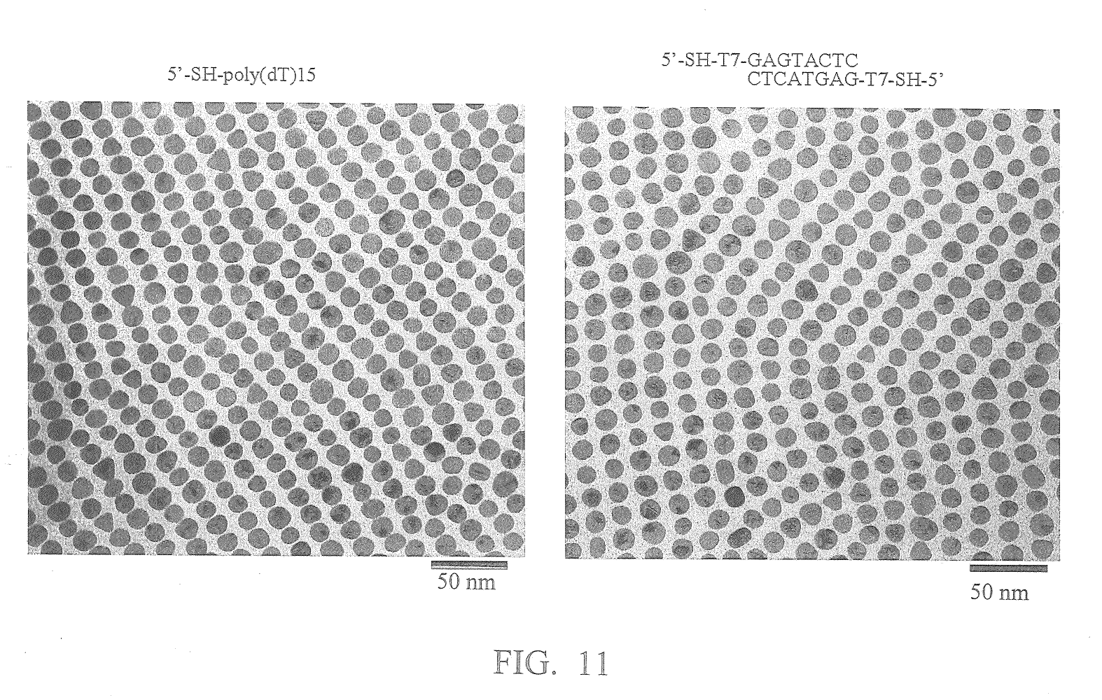

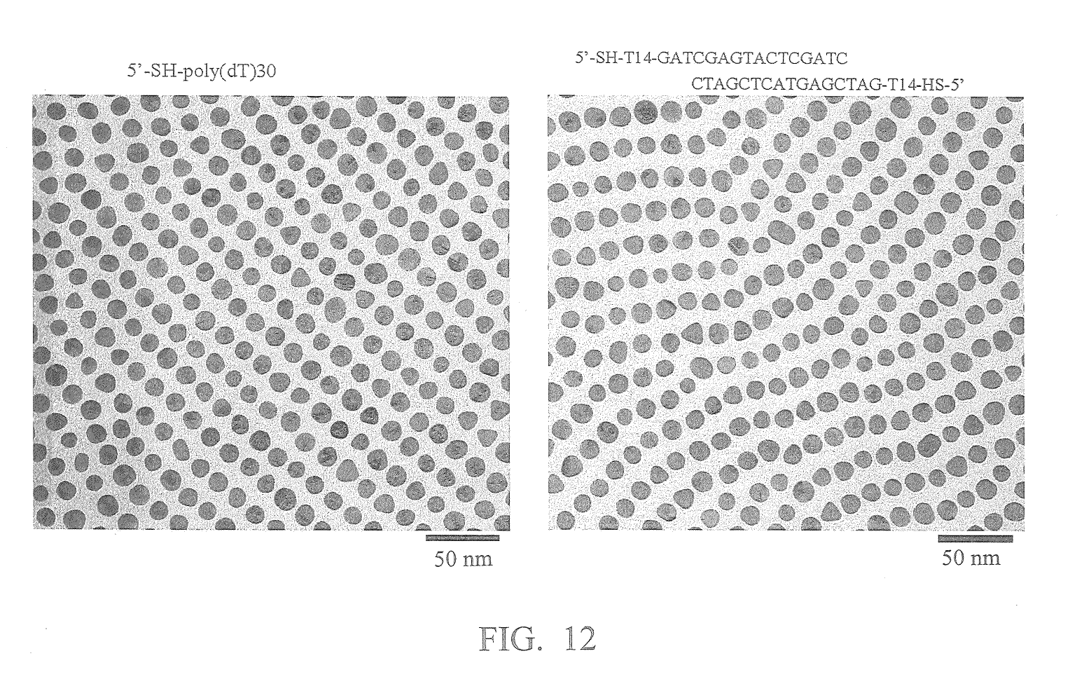

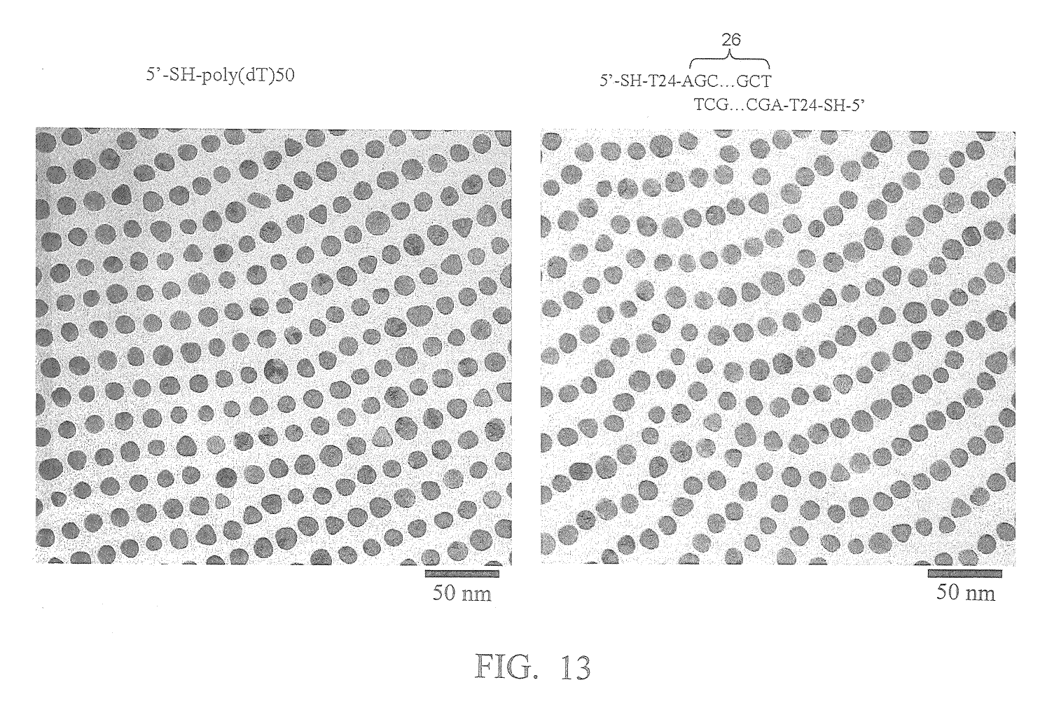

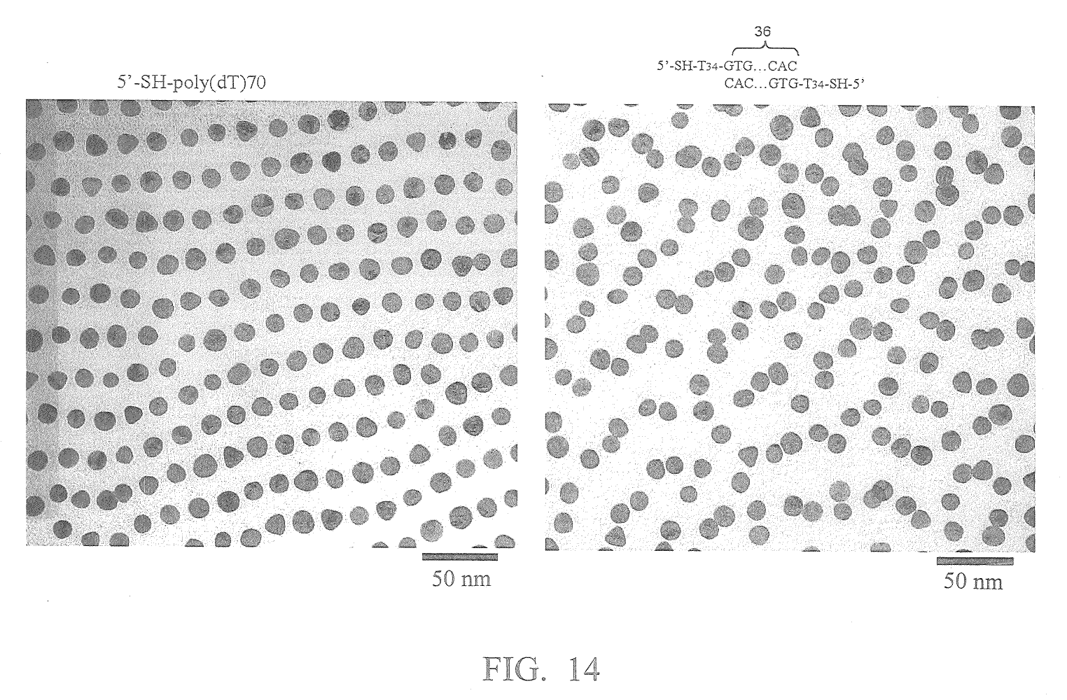

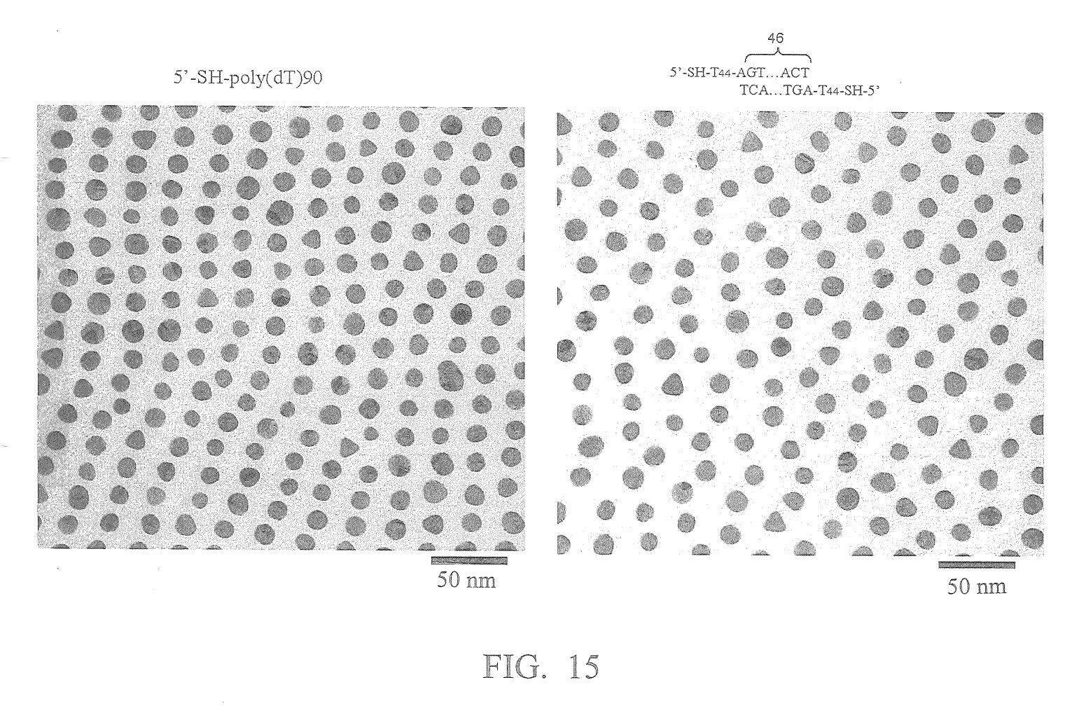

One feature of the disclosed device and method is that the nucleic acids coordinated with the nanoparticles are substantially free of Watson-Crick base-pairing. By using the term “substantially free”, this disclosure contemplates that less than 20%, more preferably less than 10%, and most preferably less than 5% of the nucleic acids coordinated with each nanoparticle are associated with nucleic acids coordinated with other nanoparticles through Watson-Crick base-pairing. Counterintuitively, the substantial absence of Watson-Crick base-pairing in the disclosed device or method does not decrease the degree of organization in the disclosed array of nanoparticles. In fact, as illustrated in FIGS. 5A-5F, more organized arrays of nanoparticles are achieved in most of the disclosed devices compared to arrays of nanoparticles organized by Watson-Crick base-pairing of nucleic acids with the same numbers of nucleotides. Without wishing to be bound by any particular theory, it is contemplated that the absence of Watson-Crick base-pairing allows significantly more numbers of nucleic acids to coordinate with the nanoparticles, thereby contributing to the formation of a high degree of order. For example, the average number of nucleic acids coordinated with a nanoparticle in this disclosure may be more than 100, while the average number of nucleic acids coordinated with a nanoparticle in devices based on Watson-Crick base-pairing is around 60.

Another feature of the disclosed device and method is that the interparticle distance can be tuned within a wider range than that achieved by using alkylthiol as capping ligands. As demonstrated in Table 1 and illustrated in FIGS. 5A-5F, by extending the length of the nucleic acid ligands, e.g. increasing the number of nucleotides (from 5 to 90), the interparticle distance (edge-to-edge) can be manipulated from about 2 nm to about 27 nm. In one embodiment, the interparticle distance may be manipulated from about 2 nm to about 25 nm or even from about 3 nm to about 25 nm. Other interparticle distance ranges within the disclosed ranges are also contemplated by this disclosure. Such wide ranges of interparticle distances cannot be achieved by methods or devices that use alkylthiols as capping ligand, which are generally characterized by interparticle distances of less than 3 nm. It is important to note that length manipulation of the nucleic acids is not limited to the 5-90 nucleotides discussed above, nucleic acids with fewer than 5 or more than 90 nucleotides may also be used in view of this disclosure.

TABLE 1
Manipulation of Interparticle Distances by Varying Nucleic Acid Length

| | Minimum Interparticle | Maximum Interparticle |
|---|---|---|
| Ligand | Distance (Edge-to-Edge) | Distance (Edge-to-Edge) |
| 5′-SH-poly(dT)5 | 2.4 ± 0.5 nm | 3.8 ± 0.6 nm |
| 5′-SH-poly(dT)15 | 6.7 ± 1.1 nm | 8.9 ± 1.2 nm |
| 5′-SH-poly(dT)30 | 9.7 ± 1.3 nm | 11.0 ± 1.3 nm |
| 5′-SH-poly(dT)50 | 12.7 ± 1.1 nm | 17.0 ± 0.9 nm |
| 5′-SH-poly(dT)70 | 14.6 ± 1.1 nm | 17.2 ± 1.3 nm |
| 5′-SH-poly(dT)90 | 21.6 ± 1.7 nm | 22.2 ± 4.6 nm |

Turning to FIGS. 6A-6C, TEM micrographs and microabsorption spectra of arrays of gold nanoparticles coordinated with different nucleic acids (5′-SH-poly(dT)₅, 5′-SH-poly(dT)₃₀, and 5′-SH-poly(dT)₉₀, respectively) supported by a 2000-mesh copper grid (7×7 μm holes) are illustrated. Again, the interparticle distances increased with the length of the nucleic acid ligands. In addition, the microabsorption spectra clearly indicate a shifting of peak absorption toward a lower wavelength, which corresponds to the observed change in the superlattice structure of the nanoparticles by the TEM.

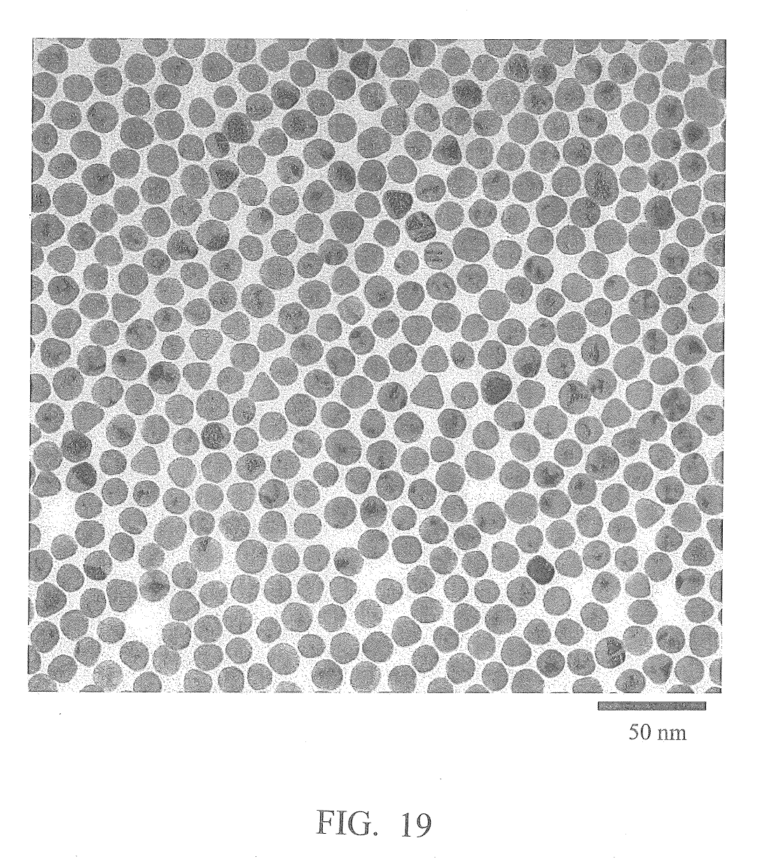

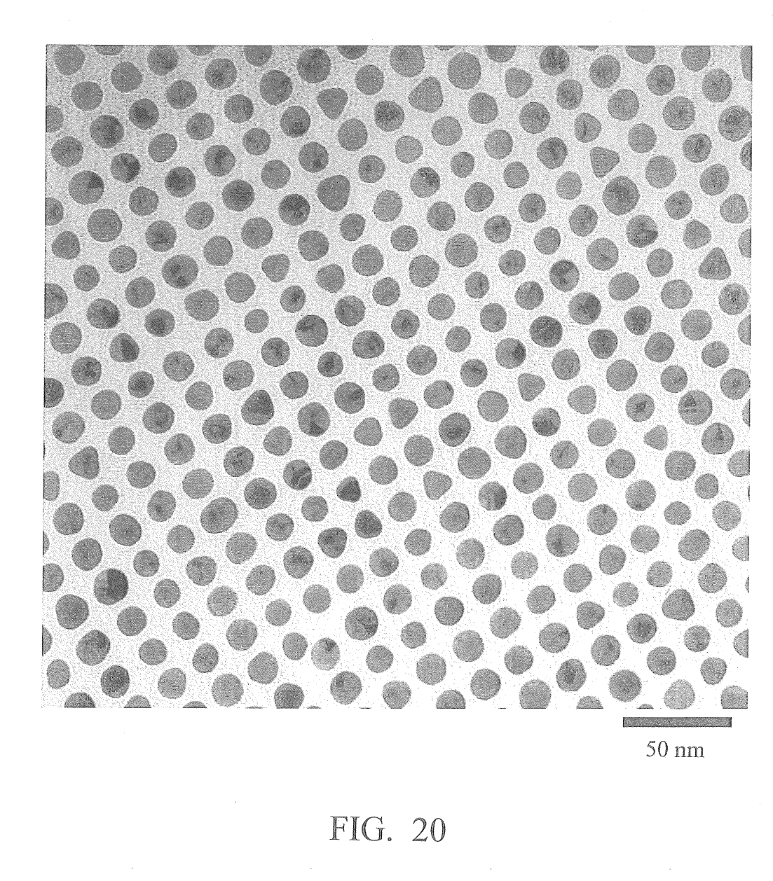

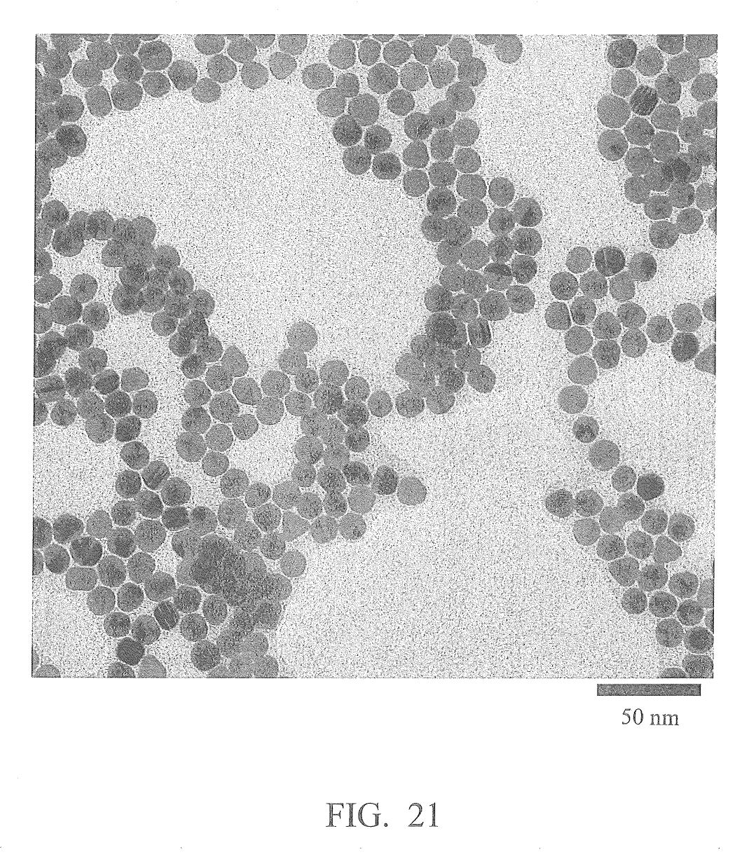

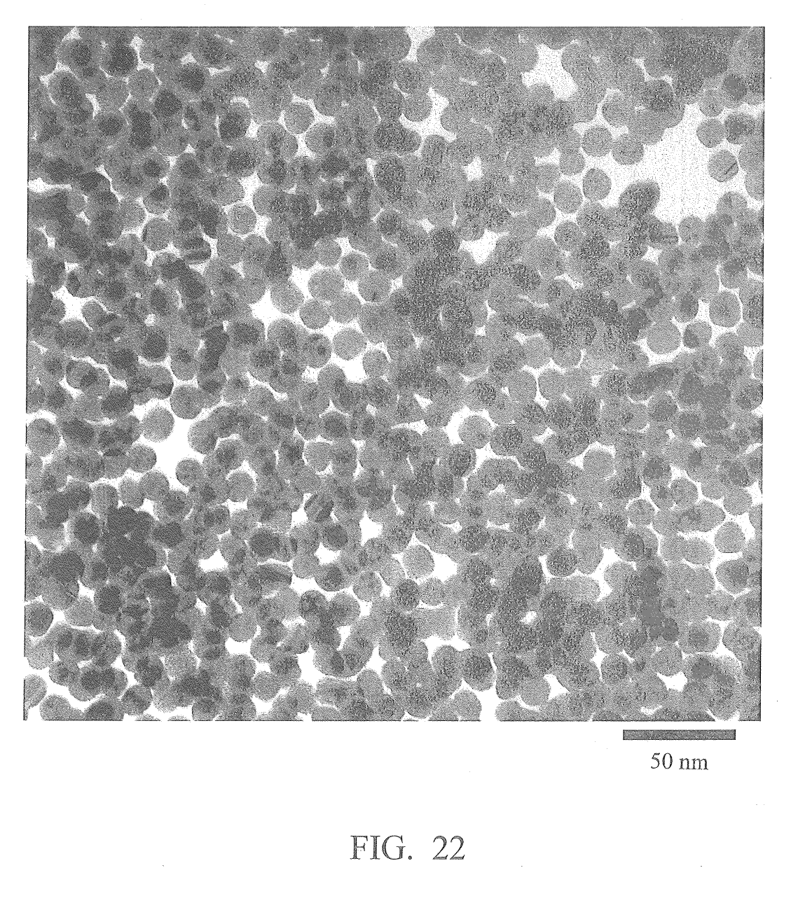

As discussed earlier, the well-organized superlattice structure achieved by the disclosed devices and methods may be related to the number of nucleic acids coordinated to the nanoparticle. To that end, FIGS. 7A-7D illustrate the degrees of organization in arrays of gold nanoparticles prepared with different molar ratios between the ligands and nanoparticle. In particular, the comparison suggests that a higher number of nucleic acids coordinated with the nanoparticle corresponds to a more organized superlattice structure. Accordingly, in one embodiment of this disclosure, the molar ratio of the ligands and nanoparticle is at least 100:1. In another embodiment, the molar ratio of the ligands and nanoparticle is from about 200:1 to about 300:1. In yet another embodiment, the molar ratio of the ligands and nanoparticle is at least 500:1 or even at least 1000:1.

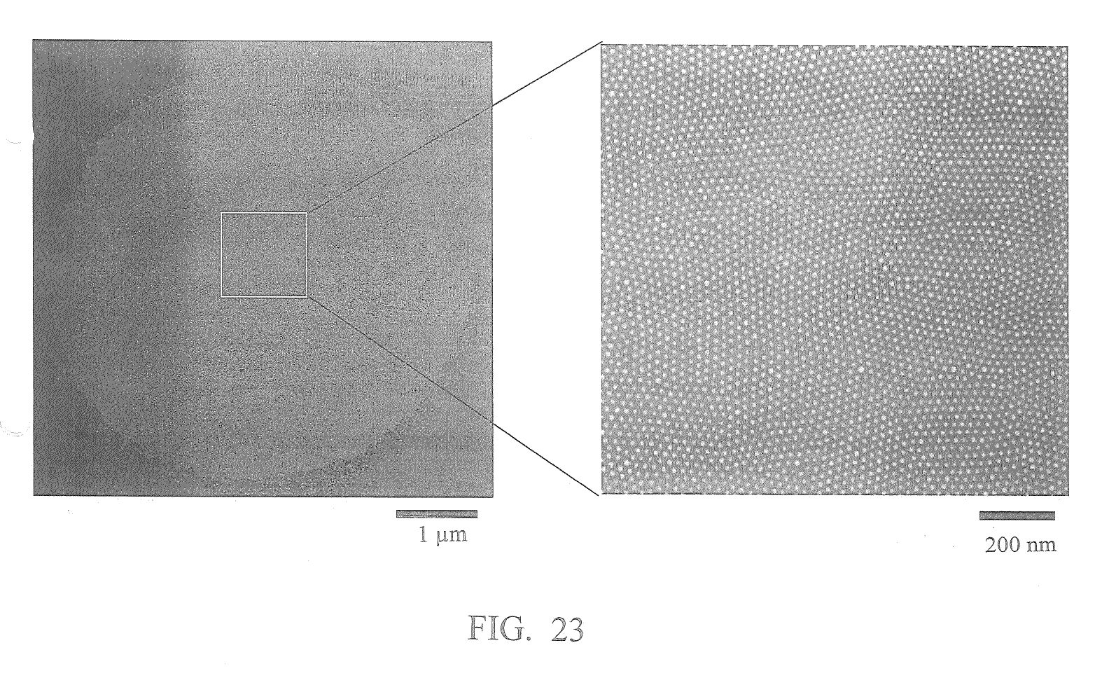

In addition to 2D superlattice structures, the array of nanoparticles disclosed herein may also form 3D crystal structures. As illustrated in FIG. 8, an array of gold nanoparticles (5′-SH-poly(dT)₅ as ligands) forms well-defined 3D crystals on a silicon substrate. The crystal thus formed may have an anisotropic optical response.

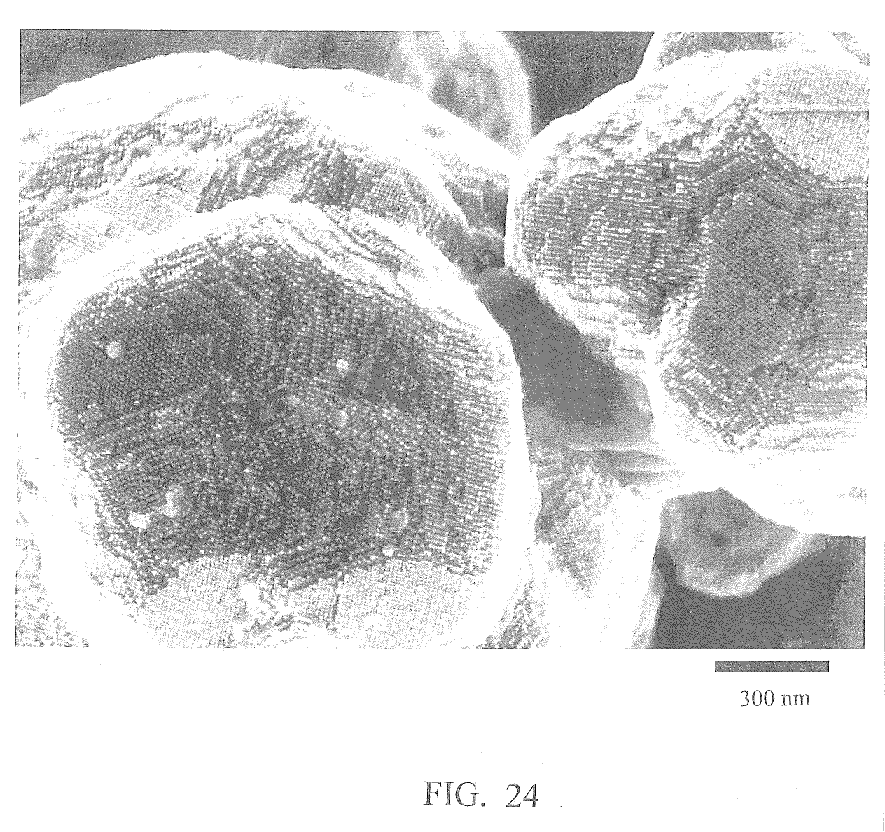

The disclosed nucleic acids-coordinated nanoparticles may be processed into micro- and nano-scale patterns by PDMS microcontact printing. As illustrated in FIG. 9, a micro-disc of 2D gold nanoparticle superlattices is formed by PDSM microcontact printing.

In one embodiment, PDMS surface pattern edges are nucleation sites of the nanoparticles to achieve line resolution with single particle size width. In another embodiment, the nanoparticles according to this disclosure are printable by a nanopen. In a refinement, micro-scale letters from gold nanoparticles can be obtained with a density of 9×10⁴/cm².

The organization of nanoparticles by using nucleic acids as capping ligands rather than as interparticle connection not only allows for formation of highly stable 2D and 3D superlattices in dewetted conditions, it also allows for more comprehensive control of those supperlattice. In particular, the nanoscale structure of the superlattices can be regulated via nucleic acids and the overall shape of the superlattices can be controlled by the micrometer-sized molds. Moreover, the disclosed nanodevice may be substrate-less, in which the superlattices may be self-supported, such as those suitable for use in foldable electronics.

While only certain embodiments have been set forth, alternative embodiments and various modifications will be apparent from the above descriptions to those skilled in the art. These and other alternatives are considered equivalents and within the spirit and scope of this disclosure.

What is claimed is:

1. A self-assembly nanodevice, comprising:
an array of nanoparticles, each nanoparticle being coordinated with a plurality of nucleic acids that are substantially free of Watson-Crick base-paring with nucleic acids coordinated with other nanoparticles.

2. The self-assembly nanodevice of claim 1, wherein the nanoparticle comprises a transition metal.

3. The self-assembly nanodevice of claim 2, wherein the transition metal is selected from a group consisting of Au, Ag, and Cd.

4. The self-assembly nanodevice of claim 1, wherein the nanoparticle comprises a quantum dot.

5. The self-assembly nanodevice of claim 1, wherein the nucleic acids are selected from a group consisting of DNAs, RNAs, PNAs, LNAs, GNAs, TNAs, and mixtures thereof.

6. The self-assembly nanodevice of claim 1, wherein the nucleic acids are DNAs selected from a group consisting of single stranded DNAs, double stranded DNAs, hairpin DNAs, dendrimer DNAs, quadruplex DNAs, and mixtures thereof.

7. The self-assembly nanodevice of claim 1, wherein the molar ratio of the nucleic acids and nanoparticle is at least 100:1.

8. The self-assembly nanodevice of claim 7, wherein the molar ratio of the nucleic acids and nanoparticle is from about 200:1 to about 300:1.

9. The self-assembly nanodevice of claim 1, wherein the average distance between two adjacent nanoparticles is from 2 nm to 27 nm.

10. The self-assembly nanodevice of claim 1, wherein the array of nanoparticles forms a supra-crystal with an anisotropic optical response.

11. A method of forming an array of nanoparticles, the method comprising:
dispersing a plurality of nanoparticles in an aqueous carrier to form a dispersion, each nanoparticle being coordinated with a plurality of nucleic acids; and
drying the dispersion, the nucleic acids coordinated with each nanoparticle being substantially free of Watson-Crick base-pairing with nucleic acids coordinated with other nanoparticles after drying.

12. The method of forming an array of nanoparticles of claim 11, wherein the nanoparticle comprises a transition metal.

13. The method of forming an array of nanoparticles of claim 12, wherein the transition metal is selected from a group consisting of Au, Ag, and Cd.

14. The method of forming an array of nanoparticles of claim 13, wherein the nanoparticle comprises a quantum dot.

15. The method of forming an array of nanoparticles of claim 14, wherein the nucleic acids are selected from a group consisting of DNAs, RNAs, PNAs, LNAs, GNAs, TNAs, and mixtures thereof.

16. The method of forming an array of nanoparticles of claim 11, wherein the nucleic acids are DNAs selected from a group consisting of single stranded DNAs, double stranded DNAs, hairpin DNAs, dendrimer DNAs, quadruplex DNAs, and mixtures thereof.

17. The method of claim 11, wherein the molar ratio of the nucleic acids and nanoparticle is at least 100:1.

18. The method of claim 17, wherein the molar ratio of the nucleic acids and nanoparticle is from about 200:1 to about 300:1.

19. The method of forming an array of nanoparticles of claim 11, wherein the average distance between two adjacent nanoparticles is from 2 nm to 27 nm.

20. The method of forming an array of nanoparticles of claim 11, wherein the array of nanoparticles forms a crystal with an anisotropic optical response.
